(19) United States
(12) Patent Application Publication
(10) Pub. No.: US 2007/0179342 A1
(43) Pub. Date: Aug. 2, 2007

(54) Wireless Laryngoscope with Internal Antennae and One Piece Construction Adapted for Laryngoscopy Training

(75) Inventors: Charles G. Miller, Allison Park, PA (US); Vince Kok-Ying Sha, Monroeville, PA (US)

Correspondence Address: BLYNN L. SHIDELER;THE BLK LAW GROUP, 3500 BROKKTREE ROAD, SUITE 200, WEXFORD, PA, 15090, US

(73) Assignees: KB PORT LLC, Allison Park, PA (US); C&V Innovations, Inc., Allison Park, PA (US)

(21) Appl. No.: 11/622,446

(22) Filed: Jan. 11, 2007

Related U.S. Application Data
(60) Provisional application No. 60/758,495, filed on Jan. 12, 2006.

(51) Int. Cl. A61B 1/267

(52) U.S. Cl. 600/188; 600/199

(57) ABSTRACT

A wireless laryngoscope has a first and a second handle portion coupled together defining an internal cavity and combining to form a handle assembly. The laryngoscope further includes a first and a second blade portion coupled together and defining an internal cavity in at least a portion thereof and combining to form a blade assembly. A light source within the internal cavity of the blade assembly illuminates at least a portion of the blade assembly, and a camera mounted within the internal cavity of the blade assembly obtains images of the operation of the laryngoscope. A transmitter is coupled to the camera and is mounted within one internal cavity with an antenna mounted within one internal cavity coupled to the transmitter, wherein the transmitter wirelessly transmits the video images of the camera to a remote receiver.

### RELATED APPLICATIONS

This application claims the benefit of U.S. Provisional patent application Ser. No. 60/758,495 filed Jan. 12, 2006 entitled “Wireless Laryngoscope.”

### BACKGROUND OF THE INVENTION

1. Field of the Invention

The present invention relates to a wireless laryngoscope and camera system, and more particularly to a wireless laryngoscope with internal antenna and one piece construction that is particularly well suited for laryngoscopy training.

2. Background Information

Dr. Richard M. Cooper, BSc MSc MD FRCPC, from the Department of Anesthesia and Pain Management, Toronto General Hospital, University of Toronto, Toronto, Ontario, Canada has eloquently introduced the need and purpose for laryngoscope noting that “man's assumption of an upright posture, coupled with our tendency to live in social groups has resulted in some bad habits—simultaneous eating and talking. This has necessitated exclusion of the larynx from the line of sight connecting the mouth to the esophagus. While this does make eating safer and more interesting, it has complicated the task for airway managers.”

The early need for laryngeal visualization was surgical. As a medical student, Benjamin Guy Babington created a “glottiscope,” in 1829. A two pronged tool, one prong (or shank) depressed the tongue while the other was positioned along the palate, reflecting sunlight for illumination of the glottis. It is unclear whether Babington actually saw the glottis, but his device was later termed a laryngoscope by his contemporary, Thomas Hodgkins. Babington was famous for his many contributions to medicine, but he never published any of his observations in the field of laryngology.

In 1844, John Avery, a surgeon at London's Charing Cross Hospital developed a head-mounted mirror that reflected candlelight onto a mirror housed within a speculum. He also didn't report his findings.

Manual Garcia (1805-1906), a professor of singing at the Royal Academy of Music in London is generally credited with the discovery of laryngoscopy. In 1854, while strolling in Paris, he saw the sun's image reflected in a store windowpane. He purchased a dental mirror for six francs and used this, in combination with a hand-held mirror reflecting sunlight, to visualize his own larynx and trachea during inspiration and vocalization. He accomplished what those before him were unable to do, largely because of his vocal control and absent gag reflex. His discovery, which he termed “autolaryngoscopy” was presented to the Royal Society in May 1855. Garcia's real interest was to better understand the organ capable of creating such a range of sounds. In 1862 he was granted an honorary medical degree and subsequently invested with many international distinctions. At the age of 100, in 1905 he was honored by the most prominent laryngologists of his time as the Father of laryngology.

Ludwig Türck, a Viennese neurologist used a technique similar to Garcia's, though apparently unaware of the singing teacher's activities. He used self-made mirrors and performed laryngoscopy on his gagging patients until the autumn sun's diminished intensity forced him to abandon his efforts. Johann Czermak, a physician and physiologist from Budapest, using a table lamp and mirrors borrowed from Türck, performed laryngoscopy. Czermak published and demonstrated his findings widely. He initially acknowledged Türck's contribution, but subsequently withdrew this. What followed was a protracted public debate, referred to as the “Türckish war” about who first used laryngoscopy for diagnostic purposes.

A laryngology clinic was established in Vienna in 1870 and minor surgical procedures were performed under visual control. In the days prior to local anesthetics, patients had to be trained to suppress their gag reflexes. Morell Mackenzie learned laryngoscopy from Czermak and went on to found London's first throat hospital, however, the techniques of indirect laryngoscopy were not used to facilitate tracheal intubation.

William Macewen, a British surgeon, was the first to intubate the larynx for surgical purposes. He practiced blind, digital intubation on cadavers and eventually employed this technique to perform a composite resection in 1878. Joseph O'Dwyer, a pediatrician raised in London, Ontario, worked at the Foundling Asylum in New York City, where he developed instruments to enable tracheal intubation which saved the lives of hundreds of children suffocating from diphtheria. Hans Kuhn modified O'Dwyer's instruments and created a long, flexible metal endotracheal tube and introducer but the technique still depended upon blind insertion, largely because light sources were inadequate to permit progress in direct laryngoscopy.

In 1895, Alfred Kirstein learned of an inadvertent tracheal insertion of an esophagoscope, and proceeded to develop a rigid laryngoscope with transmitted light. This consisted of a lamp within the handle, focused on a lens and redirected through the laryngoscope by a prism. Chevalier Jackson subsequently modified Kirstein's laryngoscope by providing distal illumination with a tungsten bulb. In 1913, Henry Janeway devised an open-sided laryngoscope with battery operated distal illumination, specifically for endotracheal intubation.

In 1941, Robert Miller introduced a new, longer, lower profile laryngoscope blade (a straighter blade), designed to pick up the epiglottis. This blade required limited mouth opening but also left little space to manipulate the endotracheal tube (ETT). Two years later, Robert Macintosh described a curved blade, designed to elevate the epiglottis by exerting its force on the base of the tongue. He believed that reducing contact with the epiglottis would be less stimulating and provide more room for manipulation of the ETT. The “Miller blade” and the “Mac blade” or “Macintosh Blade” continue to dominate the field of laryngoscopy and these represent more than 95% of the laryngoscopic blades used in practice. The proper function of both a Macintosh and Miller blade is dependent on using an appropriate length of blade. The Macintosh blade must be long enough to put tension on the glossoepiglottic ligament, and the Miller blade must be long enough to trap the epiglottis against the tongue. Both blade types are made in various designated sizes (but the overall distinctive shape is as described above). Thus, in some patients, it may be appropriate to change the length of the conventional Mac or Miller blade in order to obtain proper blade function. The changing of the length can be through replaceable blades that is common in laryngoscopes or through selecting a separate laryngoscope altogether.

In some patients, a Macintosh blade may provide a superior view or intubating conditions than a Miller blade, and vice versa. A Macintosh blade is generally regarded as a better blade whenever there is little upper airway room to pass the ET (e.g., small narrow mouth, palate, oropharynx), and a Miller blade is generally regarded as a better blade in patients who have a small mandibular space (anterior larynx), large incisors, or a long, floppy epiglottis.

A study that examined airway problems in over 18,500 adult non-obstetrical patients, direct larynoscopy was the first choice 98% of the time. Among these patients, the failure rate was 0.3% and “awkward” or “difficult” in 2.5% and 1.8% respectively. The study recognized that difficulties involving laryngoscopy and intubation are poorly described and proposed an intubation “difficulty score”. No difficulties were encountered in 55% of adult patients; minor intubations difficulties were encountered in 37%; two or three laryngoscopies were required in 9% of cases and more than three attempts were required 3% of the time. However, even “non-difficult” endotracheal intubation may be associated with airway injury. One analysis involving 266 incidents of airway injury found that 80% of laryngeal injuries occurred when laryngoscopy and intubation was thought to have been easy.

The inability to see the larynx generally results in multiple or prolonged laryngoscopic attempts with increasing force, and is associated with esophageal, pharyngeal and dental injury, arterial desaturation, hemodynamic instability and unplanned intensive care unit admissions.

More recently, compact, robust, high-resolution videochips have become available which can be embedded within laryngoscopes. These devices provide an alternative laryngeal view. These devices permit simultaneous viewing by mentor and supervisor and have been thought to accelerate the instruction of laryngoscopy. These images can be captured and replayed for analysis to further expedite and improve training. The video or static images may be useful for research, teaching or clinical documentation. Also, these devices can enable visualization in settings that would otherwise be challenging or not possible. Additionally, it has been asserted that since tissues do not have to be compressed and distracted to achieve a line-of-sight, there may be less stress and trauma to the patient during laryngoscopy; and further that, positioning should not impact upon the laryngeal view.

Several different laryngoscopes with associated camera systems have been commercialized to some degree or another, with each system allowing for indirect viewing of obstructed airways. All of these systems rely on standard wired camera technologies to provide the intubator and other medical personnel with an indirect visualization of the field on view. The digital images from these commercial camera systems are transmitted via cable to an external monitor.

The inherent weaknesses of the systems using external viewing displays are that the cables connecting the camera, to the display, limits the movement of the intubator, which may complicate an already difficult procedure. An attached cable limits the working space for medical personnel and can also cause another potential hazard. Also, having exposed cabling leaves the system susceptible to fluids damaging the sensitive electronic systems no matter how well sealed. Furthermore, cables are easily damaged from over extension, frequent use, and any number of other factors adding a substantial point of failure to the entire system.

Wireless transmitters for such systems have been proposed that could, in theory, alleviate the problems encountered with cabled camera systems. See for example U.S. Patent Application Publication 2003/0195390 and U.S. Pat. No. 6,840,903. In both these systems the cable is replaced with an external antennae attached to a transmitter. The external antennae in each of these proposed wireless systems add a separate obstruction on the laryngoscope for the user. Further, as noted above, a significant advantage for the use of camera systems in laryngoscopes is for teaching and training purposes. Both of these prior art camera systems are directed to “specialized” blade shapes (non Miller or Mac styles), and promote the advantages of such unique blades. The inventors of the present invention believe that training on such specialized blades is not useful and possibly counter productive. Having trainees gain proficiency on a blade design they are not likely to see in the actual use is less desirable (and possibly counter productive) than having them gain proficiency on conventional blade designs. Within the meaning of this application the Mac blades (AKA Macintosh blades) and the Miller blades, as known in the art, are conventional blade designs.

### SUMMARY OF THE INVENTION

It is one object for this invention to provide a wireless laryngoscope for remote viewing and capable of serving as an intubation instrument, for standard intubations and complicated intubations where the field of view is obstructed from the intubator and/or other medical staff.

A further objective is to provide a laryngoscope, which is similar in design and functionality to existing blade and handle shapes so that the intubator is familiar with its application, and such that the laryngoscope is particularly well suited for training applications.

A further objective is to provide an electronic laryngoscope with a self-contained wireless digital camera embedded within the laryngoscope, which provides real-time indirect viewing of the field of view that is also self-contained, light weight, and portable. This image will be transmitted wirelessly to its receiver and can be viewed on any video type display.

This invention will have none of its wired predecessor's weakness and all of their strengths providing a more effective instrument for use in intubations. Not being tied down by cables, the intubator will retain his full range of movement and can use the invention as he would any standard laryngoscope. In fact gaining proficiency with the present invention will presumably lead to added proficiency with conventional non-camera based laryngoscopes (except for the added visualization that is possible with camera systems). Furthermore, the video viewing display can be setup anywhere within transmission distance to the invention and then broadcast to one or multiple locations for viewing, leaving the workspace clear.

One embodiment of the present invention provides a wireless laryngoscope having a first handle portion and a second handle portion coupled to the first handle portion and defining an internal cavity, wherein the first handle and the second handle portions combine to form a handle assembly. The laryngoscope further includes a first blade portion and a second blade portion coupled to the first blade portion and defining an internal cavity in at least a portion thereof, wherein the first blade portion and the second blade portion combine to form a blade assembly. A light source is within the internal cavity of the blade assembly for illuminating at least a portion of the blade assembly, and a camera is mounted within the internal cavity of the blade assembly for obtaining images of the operation of the laryngoscope. A transmitter is coupled to the camera and is mounted within one internal cavity with an antenna mounted within one said internal cavity coupled to the transmitter, wherein the transmitter wirelessly transmits the video images of the camera to a remote receiver.

In one aspect of the invention the first blade portion is formed integral with the first handle portion and the second blade portion is formed integral with the second handle portion. In one aspect of the invention the blade assembly is one of a Miller blade and a Macintosh blade. In one embodiment of the invention the camera sends a video signal from the blade assembly to the handle assembly, and the transmitter and antenna are mounted within the handle assembly.

In one aspect of the invention a method of training laryngoscopy is provided comprising the steps of: providing a wireless training laryngoscope; and recording trainee intubation attempts using the training laryngoscope. The training laryngoscope comprising a handle assembly, a blade assembly, wherein the blade assembly is one of a Miller blade and a Macintosh blade, a light source coupled to the blade assembly for illuminating at least a portion of the blade assembly, a camera coupled to the blade assembly for obtaining images of the operation of the laryngoscope, a transmitter coupled to the camera; and an antenna coupled to the transmitter, wherein the transmitter wirelessly transmits the video images of the camera to a remote receiver. The method may further provide that at least some of the intubation attempts using the training laryngoscope are performed on simulators.

These and other advantages of the present invention will be clarified in the brief description of the preferred embodiment taken together with the drawings in which like reference numerals represent like elements throughout.

### BRIEF DESCRIPTION OF THE DRAWINGS

FIGS. 1 and 2 are front perspective views of a wireless laryngoscope with internal antennae and one piece construction that is particularly well suited for laryngoscopy training in accordance with one aspect of the present invention;

FIG. 3 is a rear side view of the wireless laryngoscope of FIGS. 1-2, with a rear handle and blade portion removed, and schematically illustrating the remote monitor of the camera system; and

FIG. 4 is a rear side view of the wireless laryngoscope of FIGS. 1-2, with a rear handle and blade portion removed.

### DESCRIPTION OF THE PREFERRED EMBODIMENTS

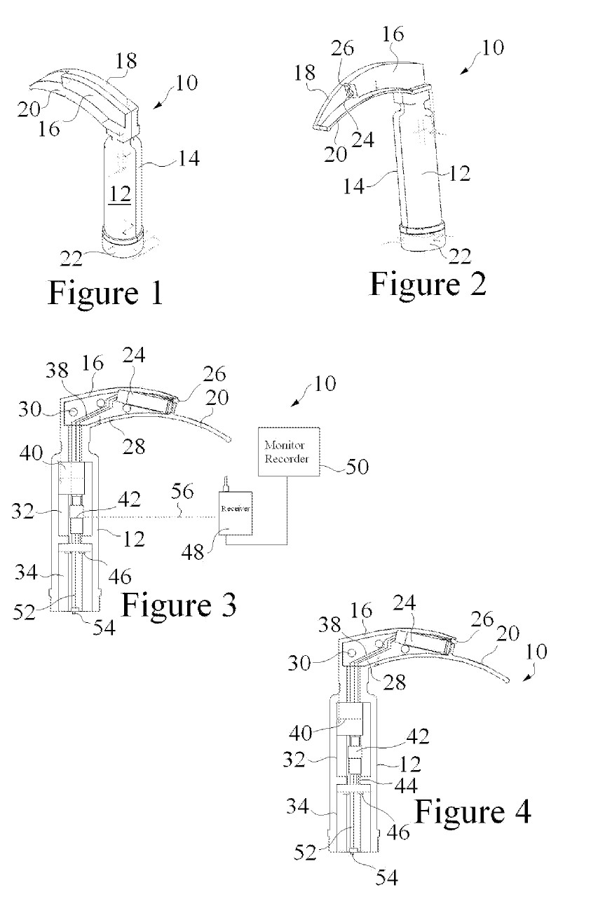

FIGS. 1 and 2 are front perspective views of a wireless laryngoscope 10 according to the present invention. The wireless laryngoscope 10 includes a front or first handle portion 12 and a second or rear handle portion 14 coupled to the first handle portion 12 and defining an internal cavity as described below. The first handle and the second handle portions 12 and 14 combine to form a handle assembly which is intended to conform to the general size and shape of conventional laryngoscope designs. The terms front and rear are merely to differentiate the handle portions 12 and 14 for purposes of explanation only. The handle portions 12 and 14 are made from any conventional material, although injection molded thermoplastic is cost effective, particularly for training purposes. In training purposes the laryngoscope 10 will likely be used on simulators (not shown) such that the laryngoscope need not be sterilized (autoclaving or the like) between uses. Consequently for constructing a training laryngoscope 10 for use with simulators a wider range of acceptable materials may be utilized.

The laryngoscope 10 further includes a front or first blade portion 16 and a rear or second blade portion 18 coupled to the first blade portion 16 and defining an internal cavity in at least a portion thereof as described below. The first blade portion 16 and the second blade portion 18 combine to form a blade assembly including a conventional extension or tongue 20.

It is important for training purposes that the blade assembly of the present invention be formed in a conventional blade shape, specifically one of a miller blade and a Macintosh blade. As shown the blade assembly is a Macintosh blade, specifically a “Mac 3” as shown. The Mac blades and the Miller blades are consider the conventional blade designs within this application. The conventional blade design is preferred even if the associated camera system allows, or even suggests as some have postulated, for an alternative blade configuration. For training purposes it is desired that the intubators gain proficiency with a style that they will likely utilize in the field (and which is likely NOT to have camera system associated therewith).

The laryngoscope 10 still provides all the advantages of a camera laryngoscope discussed above and can easily be utilized in clinical application, but the laryngoscope 10 has particular training advantages as described herein.

The blade portions 16 and 18 are made from any conventional material, although injection molded thermoplastic is cost effective, particularly for training purposes. Further as illustrated in the figures, it is possible to easily construct the front handle portion 12 and the front blade portion 16 as an integral molded unit and the rear handle portion 14 and the rear blade portion as an integral molded unit. This simplifies construction and forms a substantially sealed laryngoscope 10. The present invention provides a final “one-piece” laryngoscope 10 because the handle assembly is integral (not separable from) the blade assembly. A separable or replaceable blade assembly would be considered a two piece construction within the meaning of this application. The “one piece” construction is believed to allow for easier construction of the internal components for the wireless system as will be evidenced below.

The distal end of the handle assembly (i.e. the end opposed from the blade assembly may further include a cap 22 that is secured thereto through threads, snap fit, press fit or the like. The cap 22 can be easily formed of a molded plastic material.

A camera 24 (with integral lens) and light source 26 are mounted within an internal cavity 28 of the blade assembly. The light source 26 may be an LED element, such as a 3 mm 300 mcd element. The light source 26 will provide illumination in a conventional fashion. The camera 24 is for obtaining images of the operation of the laryngoscope and is directed generally toward the tongue 20 as shown. The internal cavity 28 may further include mounting elements such as posts 30 that engage receiving structure (not shown) on the blade portion 18 to assist in the assemble and the structural integrity of the laryngoscope 10.

The housing assembly defines an internal cavity 32 for receipt of transmission components as will be described and a power cavity 34 for receiving the power supply for the laryngoscope 10. The power supply is a battery, such as a nine volt battery received within the cavity 34.

The camera 24 forward a video signal via wires 38 extending from the blade assembly to the handle assembly. The wires 38 extend to a wireless transmitter 40 mounted within the cavity 32. The transmitter 40 is preferably an RF transmitter and selected for suitable use in a hospital type environment. However training versions of the laryngoscope 10 may not be so restricted as such training tools are often employed outside of a more restrictive hospital environment. In the United States there are selected frequencies acceptable for wireless communications for hospital type environments. In other embodiments an optical transmission (e.g. infrared) could be used provided an appropriate line of sight is maintained between the transmission and the receiving components.

A completely internal antenna 42 is mounted within one the internal cavity 32 and is coupled to the transmitter 40, wherein the transmitter 40 wirelessly transmits the video images of the camera 24 to a remote receiver 48 for display on a remote monitor/recorder 50. More than one monitor/recorder 50 may be provided. The construction of the receiver 48 and monitor/recorder system 50 are conventional and not part of the present invention. KBPort supplies a versatile digital recording system that is appropriate for this use. The monitor/recorder 50 is considered remote because it is not coupled to the laryngoscope 10 directly. The coupling is through RF link 56 (or optical link if optical transmission is utilized).

The laryngoscope 10 further included conventional contacts 46 for engaging the battery power supply in a conventional fashion which will be slid into cavity 36 with the removal of the cap 22. Further, wires 52 extend to switch 54 that is easily accessible to the user (through removing cap 22) for turning on and off the light source 26 and camera 24 (and activating transmitter 40). It is envisioned that other controls in addition to the switch 54 may be added such as adjustments for the intensity of light or the focus of the camera (i.e. an adjustable lens). Specific details of the wiring or circuitry for the laryngoscope components will be well known to those of ordinary skill in the art and need not be discussed here in detail.

The laryngoscope 10 of the present invention provides a substantially sealed wireless laryngoscope 10 that has conventional shape for the blade and the housing that makes this laryngoscope well suited for training purposes. Gaining proficiency on this laryngoscope 10 will allow the intubator to gain proficiency on those laryngoscopes he is likely to encounter in practice (i.e. the conventional laryngoscopes with Miller or Mac blades). Further the laryngoscope 10 facilitates training by allowing mentors to view (real time or via recording) the training attempts, and the monitor 50 can be easily and conveniently placed for un-obstructed viewing by those that it would be helpful to do so. The recording of intubation attempts will allow further review and comment to facilitate learning by the intubator and others. The laryngoscope 10 is not limited to training applications as it has all the advantages of a camera system laryngoscope with the additional advantages of a wireless implementation (and non-restricting internal antenna).

Although the present invention has been described with particularity herein, the scope of the present invention is not limited to the specific embodiment disclosed. It will be apparent to those of ordinary skill in the art that various modifications may be made to the present invention without departing from the spirit and scope thereof.

1. A wireless laryngoscope comprising:
A first handle portion;
A second handle portion coupled to the first handle portion and defining an internal cavity, wherein the first handle and the second handle portions combine to form a handle assembly;
A first blade portion;
A second blade portion coupled to the first blade portion and defining an internal cavity in at least a portion thereof, wherein the first blade portion and the second blade portion combine to form a blade assembly;
A light source within the internal cavity of the blade assembly for illuminating at least a portion of the blade assembly;
A camera mounted within the internal cavity of the blade assembly for obtaining images of the operation of the laryngoscope; and
A transmitter coupled to the camera mounted within one internal cavity; and
An antenna mounted within one said internal cavity, wherein the transmitter wirelessly transmits the video images of the camera to a remote receiver.

2. The wireless laryngoscope of claim 1 wherein the first blade portion is formed integral with the first handle portion and the second blade portion is formed integral with the second handle portion.

3. The wireless laryngoscope of claim 1 wherein the blade assembly is one of a Miller blade and a Macintosh blade.

4. The wireless laryngoscope of claim 1 wherein the camera sends a video signal from the blade assembly to the handle assembly.

5. The wireless laryngoscope of claim 1 wherein the transmitter is mounted within the handle assembly.

6. The wireless laryngoscope of claim 1 wherein the antenna is mounted within the handle assembly.

7. The wireless laryngoscope of claim 1 further including a control button to activate the camera, light source and transmitter.

8. The wireless laryngoscope of claim 1 further including a remote receiver and monitor.

9. The wireless laryngoscope of claim 1 wherein the wherein the first blade portion is formed integral with the first handle portion as a molded component and the second blade portion is formed integral with the second handle portion as a molded component.

10. A wireless training laryngoscope comprising:
a handle assembly;
a blade assembly, wherein the blade assembly is one of a Miller blade and a Macintosh blade;
A light source coupled to the blade assembly for illuminating at least a portion of the blade assembly;
A camera coupled to the blade assembly for obtaining images of the operation of the laryngoscope; and
A transmitter coupled to the camera; and
An antenna coupled to the transmitter, wherein the transmitter wirelessly transmits the video images of the camera to a remote receiver.

11. The wireless training laryngoscope of claim 10 wherein the camera is mounted within the blade assembly and sends a video signal from the blade assembly to the handle assembly.

12. The wireless training laryngoscope of claim 10 wherein the transmitter is mounted within the handle assembly.

13. The wireless training laryngoscope of claim 10 wherein the antenna is mounted within the handle assembly.

14. The wireless training laryngoscope of claim 10 further including a control button to activate the camera, light source and transmitter.

15. The wireless training laryngoscope of claim 10 further including a remote receiver and monitor.

16. A method of training laryngoscopy comprising the steps of:
Providing a wireless training laryngoscope comprising a handle assembly, a blade assembly, wherein the blade assembly is one of a Miller blade and a Macintosh blade, a light source coupled to the blade assembly for illuminating at least a portion of the blade assembly, a camera coupled to the blade assembly for obtaining images of the operation of the laryngoscope, and a transmitter coupled to the camera; and an antenna coupled to the transmitter, wherein the transmitter wirelessly transmits the video images of the camera to a remote receiver;
Recording trainee intubation attempts using the training laryngoscope.

17. The method of training laryngoscopy according to claim 16 wherein at least some of the intubation attempts using the training laryngoscope are performed on simulators.
